(19) United States
(12) Patent Application Publication
(10) Pub. No.: US 2009/0270994 A1
(43) Pub. Date: Oct. 29, 2009

(54) Medial and lateral femoral implants for single-compartment knee prosthesis

(76) Inventors: Roberto Schaefer, Ciudad de Buenos Aires (AR); Martin Jorge Morhac, Provincia de Buenos Aires (AR)

Correspondence Address: JAMES C. WRAY, 1493 CHAIN BRIDGE ROAD, SUITE 300, MCLEAN, VA, 22101, US

(21) Appl. No.: 12/387,322

(22) Filed: Apr. 30, 2009

Related U.S. Application Data
(63) Continuation-in-part of application No. 11/065,294, filed on Feb. 25, 2005, now Pat. No. 7,544,210.

(30) Foreign Application Priority Data
Feb. 27, 2004 (AR) P20040100619

(51) Int. Cl. A61F 2/38

(52) U.S. Cl. 623/20.3

(57) ABSTRACT

A single-compartment knee prosthesis provides different femoral implants for the medial and lateral compartments. Both femoral implants feature distinct bends and slight twists following the anatomical shape of the corresponding compartment, of about 22° for the medial implant (11) and 14° for the lateral femoral implant (31). The complementary tibial plates also differ according to whether they are adapted to the medial or to the lateral compartment. In the latter case, the shape of the tibial plate (39) is substantially semicircular whereas the medial tibial plate (19) is more elongated.

### CROSS-REFERENCE TO RELATED APPLICATION

This is a continuation-in-part (C-I-P) of our pending application Ser. No. 11/065,294.

### FIELD OF THE INVENTION

This invention is related in general to orthopædia and arthroplastia; in particular to medial and lateral single-compartment knee prosthesis, as well as to femoral components thereof and tibial plates or implants for forming complete single-compartment prosthesis.

Such prosthesis are an important therapeutical option in medicine for rebuilding joints, specially in joints suffering medial or lateral unicompartimental arthrosis, as found in patients with a genu varo/arthrosic valgo or osteonecrosis, following tibial plate fractures.

### BACKGROUND OF THE INVENTION

The knee joint essentially operates under compression under the effect of gravity. Its movement features a primary degree of freedom, which is flexoextension (normally in a 160° arch) and a secondary degree of freedom which is rotation about the longitudinal axis of the leg. The latter secondary rotation is present only when the knee is bent since, when the knee is extended, the tibia becomes locked against rotation with the femur. Some mechanical play in the joint allows extra lateral movement when the knee is slightly bent but this small degree of freedom disappears when the knee is fully stretched except in some pathological cases.

Since knees have to satisfy two diverse features, which are extensive mobility when bent more than a certain angle and high stability when straight and the knee has to support the body weight with long lever arms, it is vulnerable to articular fractures and other damage.

Flexoextension, which is the main degree of freedom of the knee, is conditioned by a joint of the troclear type because of the convex shape in both directions of the two (lateral and medial) femoral condyles. Hence, the knee is a dicondylear joint in anatomical terms and a specific troclear joint in mechanical terms.

On the tibial side, the surfaces are shaped conversely over dual parallel concavely landings, the glenoids, separated by a blunt anteroposterior crest where the tibial crown is located (n.b. although the term “glenoid” is usually used in relation to the shoulder articulation, it is used herein with a similar meaning, as used also by the renowned French specialist Kapandji cit. infra). The blunt crest fitting into the intercondylear cleavage prevents axial rotation in extension.

From a functional point of view, the knee joint includes two joints, the femorotibial joint and the femoropatelar joint. The former is formed by the condyles clasping onto their glenoids in a way such that the tibial crown fits into the intercondylean cleavage. The femoropatelar is formed by two slopes of the articular surface of the knee-cap with two faces of the femoral troclea, such that the vertical blunt crest couples into the troclear cleavage.

Just from the flexoextension point of view and in a first approach only, one may imagine the knee joint like a dicondylean knee surface sliding over dual matched concave landings. However, reality is quite more complex.

When the condyle and glenoid bearing surfaces are subject to excessive or uneven wear, the joint starts operating badly, the surrounding soft tissue may swell, the area becomes painful, knee movement becomes acutely restricted and so does the amount the knee is able to bend. As an alternative to replacing the entire knee by an orthopedic joint, unicompartimental knee surgery was suggested around 1970, consisting in replacing the bearing surfaces of the damaged compartment, either the medial (internal) compartment or the lateral (external) compartment, regardless of the other, or both. Aside from being surgically less invasive, this treatment sacrifices less healthy bone matter and retains the femoro-patelar joint and the ligament structure of the knee, in addition to the colateral compartment when it is in adequate anatomical and functional conditions.

### SUMMARY OF THE PRIOR ART

U.S. Pat. No. 3,958,278 discloses an endoprosthesis adequate for uni-o dicondylean implants which may be replaced once it wears out. It uses like unicompartimental femoral components with a bicompartimental tibial plate.

U.S. Pat. No. 5,312,411 discloses a surgical instrument for machining condyles and drilling anchoring holes for unicondylar prosthesis. FIGS. 9 and 10 thereof show a prosthesis which may be implanted using this instrument.

EP patent publication No 611,559 discloses a unicompartimental knee prosthesis.

U.S. Pat. No. 6,494,914 discloses a femoral component for an orthopedic prosthesis.

In all the abovementioned prosthesis, the anteroposterior axis of the femoral implant is straight, generally perpendicular to the longitudinal axis of the femur; see FIG. 2 of U.S. Pat. No. 3,958,278, FIGS. 7 and 10 of U.S. Pat. No. 5,312,411, FIG. 1b of EP patent publication No 611,559 and FIG. 8 of U.S. Pat. No. 6,494,914.

In addition, in all known cases, like femoral implants are used in both the medial (internal) and in the lateral (external) positions, the only differentiation being a question of laterality, in other words the curvature in the cross-direction (i.e. inside-to-outside or left-right direction). That is, identical implants are used for the left leg medial femoral implant as for the right leg lateral implant, and viceversa. This exchangeability is made possible by the anteroposterior straightness of the femoral implants.

Presumably, the reason behind this straightness is not simply a desire for exchangeability but rather stems from a biomechanical conception of all these prosthesis. In other words, the joints are designed as mechanical models which copy the desired natural movements summarized hereinabove. Since the flexoextension movement is about a generally horizontal transverse joint axis, the conventional designs result in implants extending in vertical longitudinal planes, in which condyle-like convexities are situated.

Thus the movement of such an orthopædical knee is not completely natural, movements are somewhat restricted according to biomechanical models, thereby subjecting implants to wear. Implanted patients have to get accustomed to movements that feel different and generally experience some loss of comfort.

In effect, the shape of human condyles is rather complex. There is convexity both in the inside-to-outside direction of the knee and from back-to-front (anteroposterior direction). According to the well known Kapandji model shown in Fisiologia Articular, chapter II, FIG. 42, the anteroposterior curvature varies both forward and rearward from a section which Kapandji calls the “T-point”.

In addition to the inside-outside and the back-front convexities, each condyle further features a half-moonlike bend about a vertical axis parallel to the longitudinal axis of the femur, as shown in Kapandji cit., FIG. 42. That is, the main movement of the condyles lacks fixed axes, rather the movement centres travel according to loci defined by the geometry of each condyle, as may be seen in FIGS. 45 and 46 in Kapandji cit.

Attempts to balance out this deficiency have included bearing such conventional femoral implants on mobile tibial implants. WO patent publication No 02/09,623 discloses a medial lateral single-compartment knee prosthesis; more particularly a tibial implant featuring a polyethilene bearing plate for the femoral implant. The plate is mounted with an angularly-reduced degree of turning freedom in a horizontal plane (that is, generally transversal to the longitudinal axis of the tibia) over the base plate of the tibial implant which, in turn, features a typical plug arrangement, in this case comprising two pegs (one of which coincides with the pivot axis of the bearing plate), for anchoring to the tibia bone.

Single-compartment knee systems are known to be marketed by Zimmer, Inc. (U.S.A.) under an M/G designation. Its femoral components differ from the former in that they are not completely straight but feature a slight bend. However, their degrees of inclination and curvature radii are still inadequate since the design thereof privileges maintaining cross exchangeability of the femoral components (i.e., the left medial component with the external right one, and viceversa). Hence, the values of the above-stated bend and curvature radius simply attempt to average out or minimize differences but in no way take into account the natural anatomical differences between the medial and lateral condyles.

The same can be said regarding the hemi-prosthesis femoral joint element shown in FIGS. 1 and 4 of U.S. Pat. No. 5,871,541 by Bruno Gerber and assigned to Plus Endoprothetik AG (Switzerland). Although the pictures show some degree of bend, the disclosure does not dwell on this feature and rough measurements on paper show it to appear to be about 15°. Furthermore, this U.S. Pat. No. 5,871,541 cites German patent 2,550,704 to National Research Development Corp. (UK) saying that the artificial endoprosthesis imitates the geometry of the “natural” knee joint and the bearing surfaces; however the drawings of the latter German patent depict straight femoral elements, suggesting away from a bend or a twist in a “naturally” shaped femoral implant element.

U.S. Pat. No. 6,770,099 to Zimmer Technology, Inc. shows a total implant including femoral and lateral components, the posterior tips of which have distinct heights (A≠B) and curvature functions (R136≠R146) of the articulating surface.

U.S. Pat. No. 5,314,482 by Goodfellow and assigned to British Technology Group discloses a unicondylar implant having an articular surface which has a spherical surface and is convexly curved in a direction away from a cylinder axis.

U.S. Pat. No. 3,816,855 to Saleh suggests that the convex bearing surfaces of the femoral component of an artificial condyle should closely follow the “natural shaping” but then discloses artificial condyles lacking important natural shape features such as the bend in Kapandji's model.

Likewise, US patent publication 2006/25865 by Reich et al and assigned to Aesculap AG & Co. suggests that the geometry of the artificial endoprosthesis be configured very closely to the natural shape of the knee joint and the bearing surfaces. However, the '865 publication's cognition of the natural shape is limited to a spherical bearing surface mounted on a cylindrical shape, with no suggestion of a deviation other than a bend (in FIG. 6, centre-line 48′ arches away from the axis 36′, see also paragraph 51 in the left-hand column of page 3). Moreover, the '865 publication states that it is convenient for the implant to be transversely symmetrical to the cylinder axis (relative to transverse plane 36 in FIG. 1), apparently teaching away from the notion of a bend.

### SUMMARY OF THE INVENTION

The present invention stems from recognizing and addressing operation deficiencies in current conventional prosthesis and in their biomechanical conception. As the cited Kapandji model teaches, nature has oriented the condyles in diverging directions and provided the glenoids with bearing surfaces lacking straight sections in any one of the tridimensional directions.

Therefore, an object of this invention a knee prosthesis based on an anatomical conception for copying complex human knee movements more naturally.

This and other objects and advantages are achieved in single-compartment medial/lateral knee prosthesis by means of a femoral component shaped such that the artificial condyle thereof is shaped with a bend further featuring a twist such that the anterior face thereof is at an angle to the longitudinal axis of the condyle (which in turn is at an angle to the femur axis). A twist is unlike a bend in that in the latter the locus of the radii stays on a plane. The term “twist” as used herein means that, in addition to the bend, the bearing surface forms a sort of slightly spiral landing portion such that the inclination of the surface sections changes along said portion, a small distance forward from Kapandji's “T-point”, as may be observed in a real condyle.

We have discovered that the twist is an important geometrical feature enabling the condyle part of the prosthesis to bear parallel to the tibial plate (glenoid) for all degrees of flexo-extension, taking into account that the condyles diverge backwards and away from the longitudinal axis of the femoral bone. Movement in a manner so closely resembling a healthy knee articulation assures that the ligaments continuously exercise the correct degree of tension.

Preferably, since both condyles diverge in different degrees from the longitudinal axis of the femoral bone, the aforesaid spiral landing portion according to the present invention is provided in different shapes depending on whether it is a medial or a lateral implant. In other words, two distinct single-compartment femoral implants are provided for the medial and lateral compartment, which further differ according to their laterality (i.e., axial symmetry), whether they are destined to the left or the right knee. As a result, the present invention provides four distinct femoral implants which differ insofar their bend-and-twist direction and inclination for the respective four different knee compartments found in the lower members of a human being.

### BRIEF DESCRIPTION OF THE DRAWINGS

These and other features, improvements and details of the object of this invention and the manner in which it may be reduced to practice may be understood better by means of the following detailed description, by way of example only but not to restrict the scope of the invention, of embodiments shown in the attached drawings, wherein:

FIG. 1 is an elevation view of a medial femoral implant for a single-compartment knee prosthesis according to this invention;

FIG. 2 is a rear view of the medial femoral implant of FIG. 1;

FIG. 3 is a sagittal view of the single-compartment medial femoral implant of FIGS. 1 and 2;

FIG. 4 is a schematic view representing the variation of the curvature radius of the medial femoral implant of FIG. 3;

FIGS. 5A and 5B are a pair of sketches explaining the meaning and illustrating the direction of the twist feature in the femoral implants according to this invention;

FIGS. 6A, 6B and 6C are front views of a model knee articulation showing the bottom of the femoral bone and the top of the tibia bone engaged by means of single-compartment medial knee prosthesis comprising the femoral implant of FIG. 1 articulating on a matching medial tibial implant in respective fully extended, midway and fully bent positions of the knee;

FIGS. 7A, 7B and 7C are respectively alike FIGS. 6A, 6B and 6C except that the bones are engaged by means of a prior art single-compartment medial knee prosthesis;

FIG. 8 is an elevation view of a femoral lateral implant for a single-compartment knee prosthesis according to the invention;

FIG. 9 is a rear view of the femoral lateral implant of FIG. 8;

FIG. 10 is a sagittal view of the femoral lateral implant of FIGS. 8 and 9;

FIG. 11 is a schematic representing the different curvature radii of the femoral lateral implant of FIGS. 8, 9 and 10.

### TERMINOLOGY

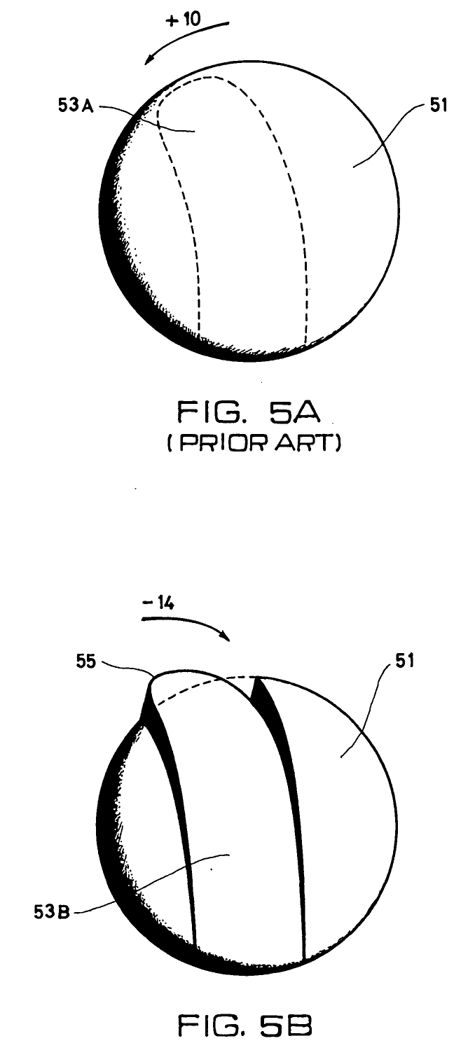

FIGS. 5A and 5B show what is meant by a bend without a twist and a bend with a twist, respectively, and are intended solely by way of clarification and not to reflect precise condyle shapes since certain assumptions are made which do not hold in the actual implant embodiments.

In FIG. 5A, a spherical surface 51 is shown having an articular portion 53A inscribed (shown in dotted lines) therein representing a virtual condyle surface. The portion 53A bends to the left about 10° as indicated but is not twisted within the meaning of the present invention, in other words there is no twist in portion 53A other than the inclination following the curvature of the sphere 51. This generally represents the design principle of the aforementioned '865 publication.

FIG. 5B sketches a similar spherical surface 51, a portion 53B of which—corresponding to the same area as the portion 53A in FIG. 5A—simulates an implant bearing area. According to the context of the present invention. The articular portion 53B has been segregated and twisted counter-spherewise about −14°, in the other direction as given by the negative sign of the twist angle, such that the upper tip 55 looks “tilted” in relation to the surface of the sphere 51.

FIGS. 5A and 5B are intended solely by way of clarification to explain the meaning of the twist at a glance but should not be construed as representing actual condyle shapes to scale; in fact the twist has been exaggerated and certain assumptions are made, such as the spherical shape of the base surface 51, which do not hold in the actual implant embodiments wherein the radii vary preferably according to the Kapandji model described in more detail further on herein. These assumptions are made to account for the difficulty in explaining a three-dimensional problem by two-dimensional visual means.

### DETAILED DESCRIPTION OF A PREFERRED EMBODIMENT

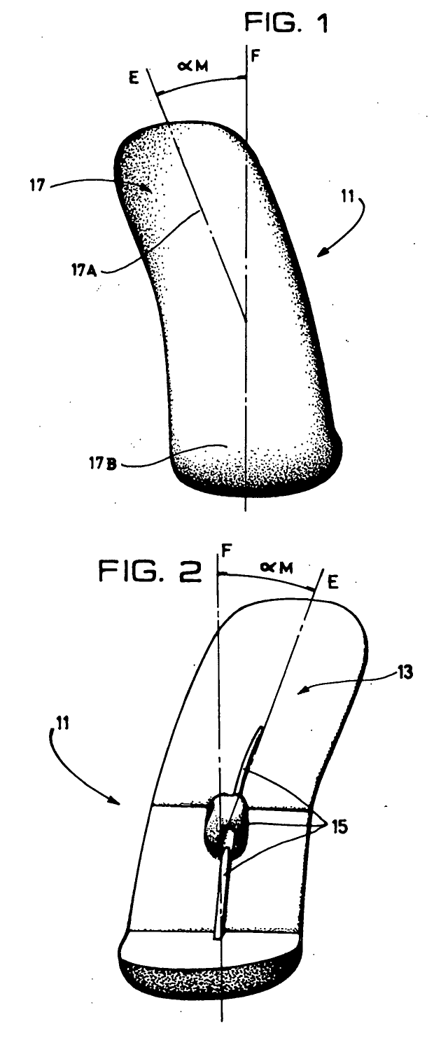

Describing in detail how the invention may be reduced to practise, the drawings depict the implants of the single-compartment knee medial-lateral prosthesis including the essential features of the present invention according to a preferred embodiment. FIGS. 1, 2 and 3 embody a femoral medial implant or implant 11 made from titanium or other suitable, preferably metallic material at least two 2 mm thick, not more than 16 to 18 mm across according to the size of the patient. Anchoring means 15 project from its inner side 13 for anchoring to the femoral bone of the patient to be implanted.

The anchorage means 15 may comprise a plug, peg or thorn and an antirotary crest (as illustrated) or alternatively two pegs as known in some conventional femoral implants such as the forementioned Zimmer's. The peg 15 is about 15 mm long and is inclined 30° from back to front and from distal to proximal.

The front face 17 forms a contact surface which is discretely convex, one part 17A of which bears on the tibial plate 19 as described further on herein when the leg is stretched and another part 17B for bearing on the tibial plate 19 when bending the knee. The convexity in the left-right direction determines a curvature radius which is about 60% greater than the width of the implant 11 or, in other words, forms an arch subtending an angle of about 35-36°, i.e. about one-tenth of a circumference. For instance, for a typical width of 20 mm, the curvature radius of the trans-verse convexity is about 32 mm, or if the width of the implant is 25 mm for larger bone types, the curvature radius is about 40 mm. The arch may be well-rounded to make the implant more tolerant or self-adjustable to slight installation variations or else be slightly flattened in the middle to conform more closely to anatomical morphology.

FIG. 4 graphs the variation of the curvature radius of the “landing” of the artificial condyle, following the cited Kapandji model. The landing is shown as a condyle profile defined by the locus of the successive contact points that bear on the tibial plate during movement in a full range between complete flexion and extension. Of course, the so-called contact point is not really a point but a surface, the contact point being considered as the contact surface centerpoint or the point of maximum instantaneous bearing pressure transmission.

Referring back again to FIGS. 1-3, the contact surface 17 is shaped with a significant bend compounded with a slight twist when seen in anteroposterior view such that, when the knee is bent 90°, the surface 17A determines an inclination to the condyle axis of about 22°, say in the range of 19° or 20° to 30°. The condyle angle is given as the natural inclination of the condyle relative to the femur longitudinal axis, about 19° in the case of the medial condyle. As explained hereinbefore with reference to FIG. 5B, the “twist” in FIG. 1 can be envisaged as the upper right-hand corner pushing slightly into the page and the upper left-hand corner conversely coming out of the drawing page towards the viewer, the degree of the resulting bend-plus-twist curve is indicated by the angle αₘ between the condyle axis F and the projection on the drawing page of the contact tangent direction E in the extended position.

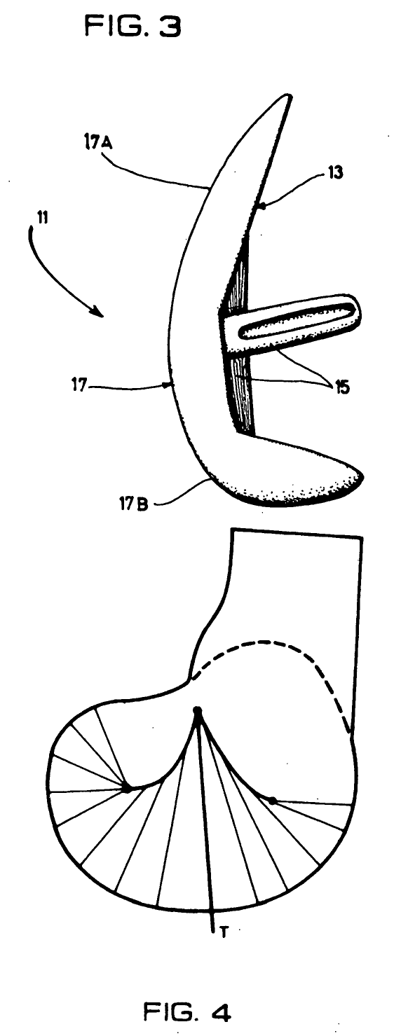

The side-view of FIG. 3 shows how the bend-and-twist of the femoral implant 11 follows the normal anatomy of the internal condyle, tracing a spiral or evolutive curve as described by Fick. The prosthesis 11 extends from the point of the condyle corresponding to the foremost one of the femorotibial bearing points (Kapandji's “T-point”) to the rear region of the condyle. The rear part 13 comprises several flat faces, as suggested by Zimmer et al, for facilitating and standardizing the surgical machining down of the condyle bone for preparing it to receive the implant 11.

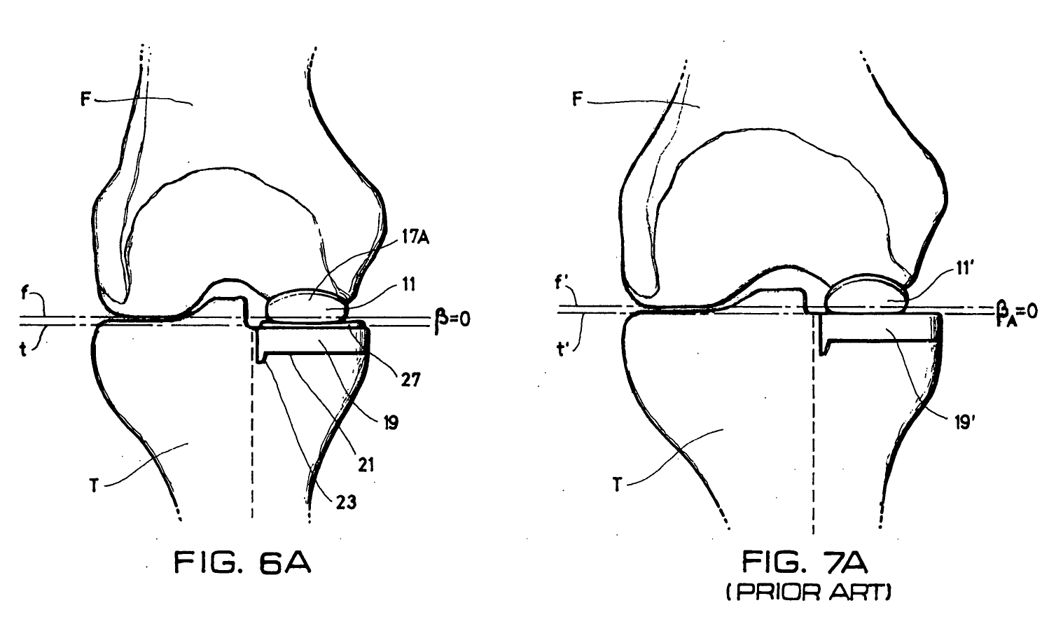

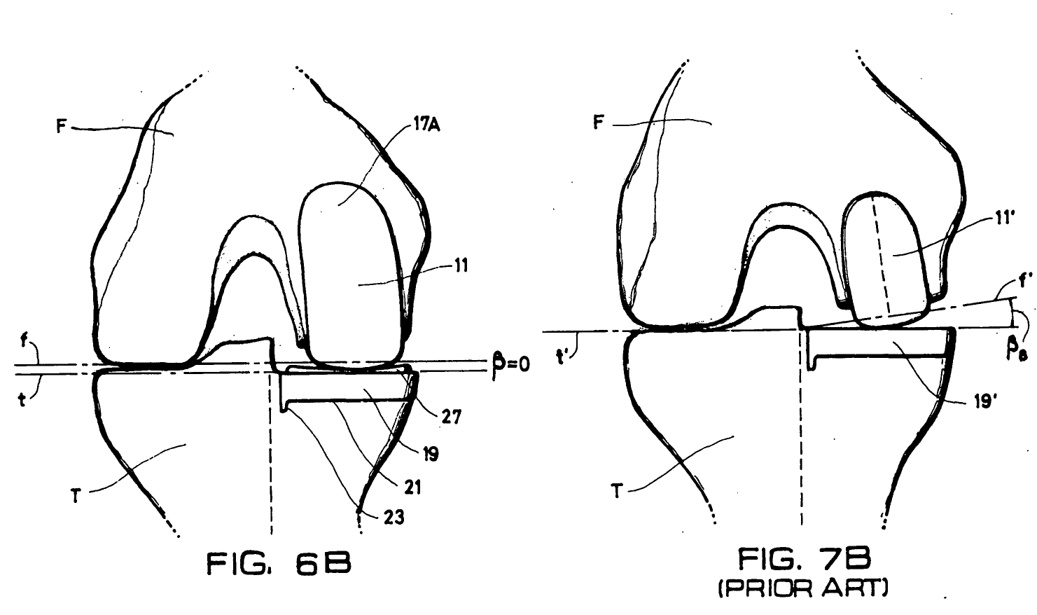

The femoral medial implant 11 is shown in conjunction with a tibial medial implant 19 in FIGS. 6A-B-C, the letter suffices (which are generally omitted for brevity sake in this specification) of the figure numbers corresponding to different states of leg extension. The tibial implant 19 comprises a metallic base 21 made of titanium, a chrome and cobalt alloy or suitable material 2 mm thick for instance. Its underface features a rough surface and one but preferably two perpendicular crests 23 for anchoring to the tibia T of the patient, affixing the implant 21 against play and relative movement of any kind, including rotation and slipping on the surface of the tibia T. The upper face of the base 21 holds a snap-in, polyethylene pad 27. The pad 27 is elongated in the anteroposterior (front-to-back) direction. Its topface is flat in the middle where the front face 17 of the condyle contact surface bears and travels along as it turns through FIGS. 6A-B-C. The pad topface is smoothly ridged upwards about 2 mm at the front and back.

An advantage of the shape of the contact surface 17 according to the invention is that the femoral implant 11 bears flat against the tibial plate 19 for any degree of knee rotation between 180°-extension (FIG. 6A) and 90°-flexion (FIG. 6C). This is indicated by the nil angle β=0° formed between the contact planes f and t corresponding to the condyle implant 11 and the glenoid plate 19, respectively. We have discovered that carefully mimicking the anatomic geometry or profile in these implants 11 and 31 without neglecting the twist in another direction, however slight it may have been considered, that accompanies the lateral bend provides a more natural movement of the knee, reduces the difficulties a patient may face until he or she assimilates the orthopedic movements and lengthens the useful lifeterm of the prosthesis. We have further found that these advantages easily compensate having to make specific femoral implants available for each one of the four compartments.

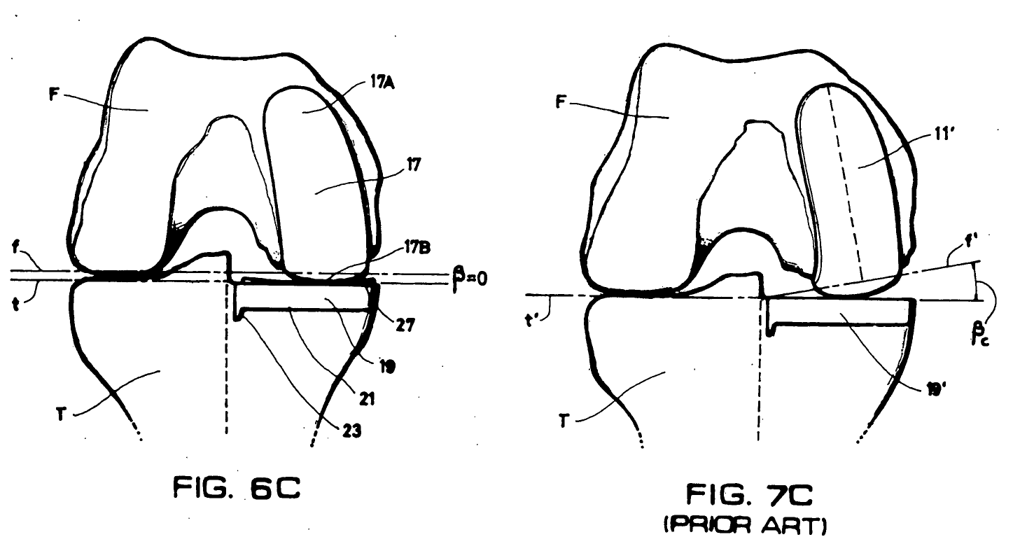

FIGS. 7A-B-C are provided for comparison to illustrate an advantage of the invention. They are the same as FIGS. 6A-B-C—that is FIG. 7A compares to FIG. 6A, FIG. 7B to FIG. 6B and FIG. 7C to FIG. 6C—except for a representative prior-art implant 11′ (such as a Zimmer model referred to beforehand) anchored to the femoral bone F instead of the femoral implant of the invention. The six FIGS. 6A-7C are instrumental in showing the improved operation achieved with a unicondylar implant 11 shaped according to the present invention.

The prosthesis in FIGS. 7A-B-C may use a complementary prior-art tibial implant 19′ or one similar to the plate 19 disclosed in the forementioned application Ser. No. 11/065,294, the pertinent disclosure of which is hereby incorporated by reference, the result is generally the same. The contact area between the implants 11′ and 19′ of the prior art prosthesis is smaller than between the implants 11 and 19 according to the invention such that the prior art femoral implant cannot bear flat against the tibial implant for all degrees of rotation, leading to greater wear. For instance, if the prior art implant 19′ is positioned to bear flat (β_{A}=0°) when the leg is extended as represented in FIG. 7A, the planes of contact f″ and t′ will begin to diverge as soon as the knee begins to bend and become quite tilted by the time the knee is half flexed as represented in FIG. 7B (β_{B}≠0°), and even more tilted by the time the knee becomes fully flexed as represented in FIG. 7C (i.e. β_{C}>β_{B}>0°).

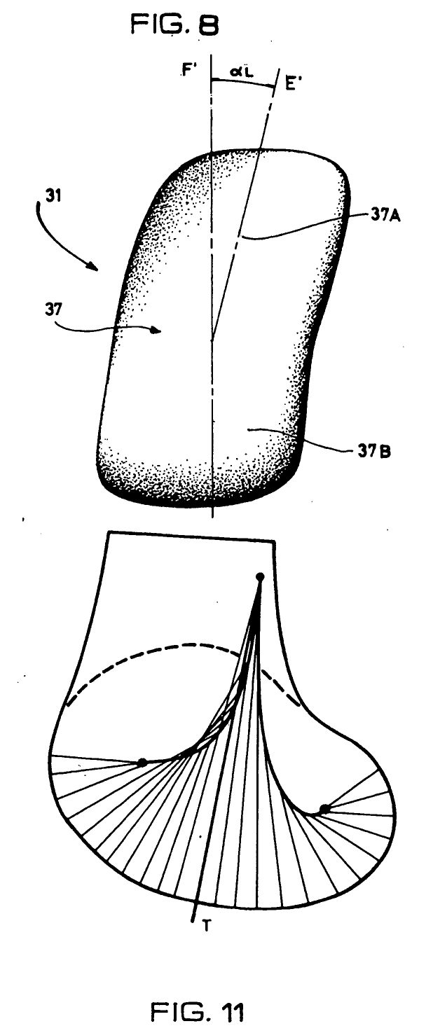

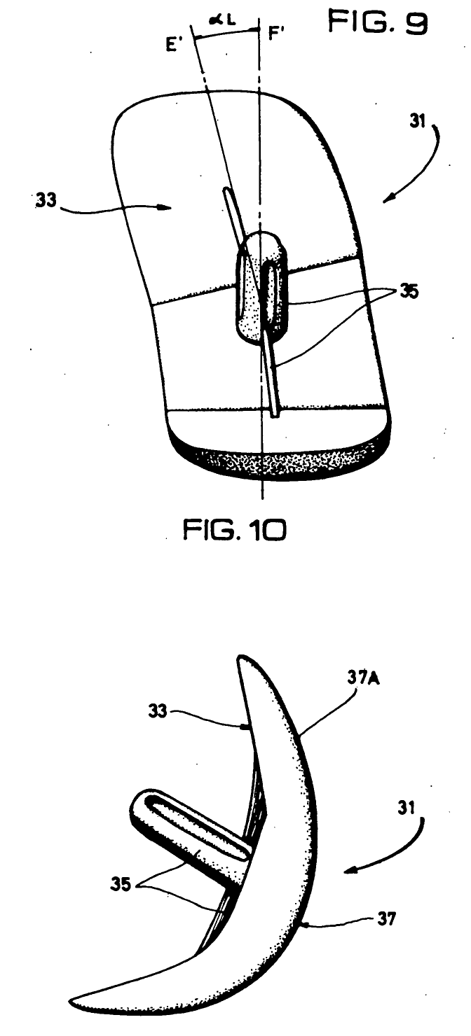

FIGS. 8, 9 and 10 show an embodiment of the femoral lateral component or implant 31 which comprises a metallic member 33 (e.g. titanium) 2 mm thick not more than 16 to 18 mm across according to the size of the patient. Means 35 project from its inner side 33 for anchoring to the femoral bone of the patient in a similar way to that described in connection with the femoral medial implant 11. The front face 37 forms a contact surface which is discretely convex, one part 37A of which bears on the tibial plate 39 (FIGS. 12-14) when the leg is straight and another part 37B which bears on the tibial plate 39 while the knee is bent.

As before, the contact surface 37 is bent and twisted in anteroposterior view such that the anterior bearing surface 37A is at an angle α_{L} of about 14°, e.g. in the range of 10° to 17°, to the condyle axis. FIG. 11 graphs the progression of the curvature radius of the “landing” of the artificial condyle, following the cited Kapandji model for the lateral condyle.

As a result, both femoral implants 11 and 31 feature different bends, according to whether each corresponds to the medial or lateral position of the knee, which is greater in the medial implant 11 than in the lateral implant 31.

The curvature, bend and twist of the implant 31 are distinct in that they closely resemble the normal anatomy of the external condyle, following the spiral or evolutive curve described by Fick. The prosthesis 11 extends from the point of the condyle corresponding to the foremost femoro-tibial bearing point (or Kapandji's “T-point”) to the rear region of the condyle.

The lateral tibial implant is different to the medial tibial implant 19 in that both the pad 47 and its metal base 41 are semicircular. The topface is generally flat and is smaller so as to allow less sliding thereon. The remaining features thereof are generally the same and disclosed in our cited application Ser. No. 11/065,294, the pertinent disclosure of which is hereby incorporated by reference.

With slight variations, the comparison provided by the six FIGS. 6A-7C illustrating the better operation of the medial implants 11 also holds for the lateral implants 11, particularly insofar keeping the femoral and tibial bones F and T in their natural orientations, and hence need not be duplicated herein. Such advantages enable a patient to maintain his or her natural leg motions and gait, extending the useful life-time of the prosthesis. We have discovered that not neglecting the twist that accompanies the lateral bend in favour of simplification as in the prior art provides substantial advantages that easily compensate having to make specific femoral implants 11 and 31 available for each one of the four compartments.

Of course, certain changes in the construction, materials, arrangement and shape of the detailed examples of the implants may be made without departing from the scope of the present invention. Dimensions of the implants may vary according to the size of each patient. In particular, the invention preferably foresees manufacturing the femoral implants 11 and 31 in six different sizes and the tibial implants 19 and 39 in five different sizes.

We claim:

1. A medial or lateral femoral implant for a single-compartment prosthesis of a knee, the femoral implant comprising a bearing surface for replacing that of the condyle of the corresponding medial or lateral knee compartment and an anchoring system for implanting the femoral implant to the femur of a patient in one of the corresponding medial or lateral knee compartments, wherein said bearing surface is provided with a shape closely following the anatomy of a normal condyle, including a predetermined bend with a twist in anteroposterior view.

2. A medial or lateral femoral implant according to claim 1, wherein the predetermined bend and twist are specific to the corresponding medial or lateral compartment of the knee.

3. A medial femoral implant according to claim 1, wherein said bearing surface includes an extension bearing portion bearing on a tibial plate at knee extension, said extension bearing portion having an inclination greater than 19° to the medial condyle axis.

4. A medial femoral implant according to claim 3, wherein said inclination is in the range of 19° to 30°.

5. A medial femoral implant according to claim 4, wherein said inclination is about 22°.

6. A lateral femoral implant according to claim 1, wherein said bearing surface includes an extension bearing portion bearing on a tibial plate at knee extension, said extension bearing portion having an inclination in the range of 10° to 17° to the lateral condyle axis.

7. A lateral femoral implant according to claim 6, wherein said inclination is about 14° to the horizontal.

8. A femoral implant according to claim 1, wherein the convexity in the left-right direction determines a curvature radius which is about 60% greater than the width of the implant.

9. A single-compartment medial knee prosthesis further comprising: the medial femoral implant of claim 3 and a medial tibial implant for bearing of the bearing member of said medial femoral implant, said tibial implant having a bearing surface closely shaped to the normal anatomy of the corresponding medial glenoid.

10. A single-compartment lateral knee prosthesis further comprising: the lateral femoral implant of claim 6 and a lateral tibial implant for bearing of the bearing member of said lateral femoral implant, said lateral tibial implant having a bearing surface closely shaped to the normal anatomy of the corresponding lateral glenoid.

11. A prosthesis according to claim 10, wherein the topface of the tibial implant(s) has a depression in which a plate of synthetic material snaps into.

12. A prosthesis according to claim 10, wherein said tibial implants are substantially flat in the cross-direction.

13. A prosthesis according to claim 9, wherein said medial tibial implant is elongated in its anteroposterior direction.

14. A prosthesis according to claim 9, wherein the underface of the tibial implant(s) has a rough surface and two anchoring crests perpendicular to one another.

15. A prosthesis according to claim 9, wherein the topface of the tibial implant(s) has a depression in which a plate of synthetic material snaps into.

16. A prosthesis according to claim 9, wherein said tibial implants are substantially flat in the cross-direction.

17. A prosthesis according to claim 1, wherein said femoral implants feature bends and inclinations which are substantially different to one another.

18. A prosthesis according to claim 1, wherein said medial and lateral femoral implants feature curvature radii which are respectively different to one another.

19. A femoral implant according to claim 1, wherein the convexity in the left-right direction determines an arch subtending an angle of about 35° or 36°.
